(19) United States
(12) Patent Application Publication
(10) Pub. No.: US 2018/0142266 A1
(43) Pub. Date: May 24, 2018

(54) A METHOD OF PRODUCING ALCOHOLS

(71) Applicants: Thomas Haas, Münster (DE); Anja Hecker, Münster (DE); Stefan Gilch, Dülmen (DE); Thomas Bülter, Duisburg (DE)

(72) Inventors: Thomas Haas, Münster (DE); Anja Hecker, Münster (DE); Stefan Gilch, Dülmen (DE); Thomas Bülter, Duisburg (DE)

(21) Appl. No.: 15/579,442

(22) PCT Filed: Jun. 10, 2016
(86) PCT No.: PCT/EP2016/063304

§ 371 Date: Dec. 4, 2017

(30) Foreign Application Priority Data
Jun. 30, 2015 (EP) 15174527.0

(51) Int. Cl. C12P 7/04; C12N 9/02; C12N 9/04; C12P 7/16

(52) U.S. Cl. CPC C12P 7/04; C12N 9/0008; C12P 7/16; C12N 9/0006; C12Y 101/01001; C12Y 102/0101

(57) ABSTRACT

There is provided a microbial cell which is capable of producing at least one higher alcohol, wherein the microbial cell is genetically modified to include an increased expression relative to its wild type cell of at least one acyl-CoA reductase (E₁₁). In particular, there is provided a microbial cell capable of producing at least one higher alcohol, wherein the microbial cell is genetically modified to include an increased expression relative to its wild type cell of at least one acyl-CoA reductase (E₁₁) and wherein the cell is capable of producing a carboxylic acid and/or ester thereof using ethanol-carboxylate fermentation.

### FIELD OF THE INVENTION

The present invention relates to a biotechnological method of producing higher alcohols from a carbon source. In particular, the method uses a recombinant cell for the production of a higher alcohol from a carbon source.

### BACKGROUND OF THE INVENTION

Butanol and higher alcohols have several uses including being used as fuel. For example, in the future, butanol can replace gasoline as the energy content of the two fuels is nearly the same. Further, butanol as an alternative fuel has several other superior properties compared to for example ethanol. These include, butanol having higher energy content, being less “evaporative” and being easily transportable compared to ethanol. Butanol is also known to be less “evaporative” compared to gasoline. For these reasons and more, there is already an existing potential market for butanol and/or related higher alcohols. Butanol and other higher alcohols are also used as industrial solvents. Higher alcohols are also used in the perfume and cosmetic industry. For example, hexanol is commonly used in the perfume industry.

Currently, butanol and other higher alcohols are primarily manufactured from petroleum. These compounds are obtained by cracking gasoline or petroleum which is bad for the environment. Also, since the costs for these starting materials will be linked to the price of petroleum, with the expected increase in petroleum prices in the future, butanol and other higher alcohol prices may also increase relative to the increase in the petroleum prices. There is thus a need in the art to find an alternative source of higher alcohol production.

Historically (1900s-1950s), biobutanol was manufactured from corn and molasses in a fermentation process that also produced acetone and ethanol and was known as an ABE (acetone, butanol, ethanol) fermentation typically with certain butanol-producing bacteria such as Clostridium acetobutylicum and Clostridium beijerinckii. This method has recently gained popularity again with renewed interest in green energy. However, the “cornstarch butanol production” process requires a number of energy-consuming steps including agricultural corn-crop cultivation, corn-grain harvesting, corn-grain starch processing, and starch-to-sugar-to-butanol fermentation. The “cornstarch butanol production” process could also probably cost nearly as much energy as the energy value of its product butanol.

The Alfol® Alcohol Process is a method used to producing higher alcohols from ethylene using an organoaluminium catalyst. The reaction produces linear long chain primary alcohols (C₂-C₂₈). The process uses an aluminium catalyst to oligomerize ethylene and allows the resulting alkyl group to be oxygenated. However, this method yields a wide spectrum of alcohols and the distribution pattern is maintained. This constant pattern limits the ability of the producer to make only the specific alcohol range that is in highest demand or has the best economic value. Also, the gases needed in the reaction have to be very clean and a distinct composition of the gases is needed for the reaction to be successfully carried out.

WO2009100434 also describes an indirect method of producing butanol and hexanol from a carbohydrate. The method includes a homoacetogenic fermentation to produce an acetic acid intermediate which is then chemically converted to ethanol. The ethanol and a remaining portion of the acetic acid intermediate are then used as a substrate in an acidogenic fermentation to produce butyric and caproic acid intermediates which are then chemically converted to butanol and hexanol. However, this method uses expensive raw materials such as carbohydrates and has two additional process steps, the formation of the esters and the chemical hydrogenation of the esters which make the method not only longer but also results in loss of useful material along the way.

Perez, J. M., 2012 discloses a method of converting short-chain carboxylic acids into their corresponding alcohols in the presence of syngas with the use of Clostridium ljungdahlii. However, short-chain carboxylic acids have to be added as a substrate for the conversion to the corresponding higher alcohol.

The currently available methods of higher alcohol production thus has limitations in mass transfer of the gaseous substrates into fermentation broth, low productivity, and low concentrations of end products, resulting in higher energy costs for product purification.

Accordingly, it is desirable to find more sustainable raw materials, other than purely petroleum based or corn based sources, as starting materials for butanol and other higher alcohol production via biotechnological means which also cause less damage to the environment. In particular, there is a need for a simple and efficient one-pot biotechnological production of butanol and other higher alcohols from sustainable raw materials.

### DESCRIPTION OF THE INVENTION

The present invention provides a cell that has been genetically modified to produce at least one higher alcohol. In particular, the cell may be capable of converting ethanol and/or acetate to at least one higher alcohol. Namely, the cell may be genetically modified to express an acyl-CoA reductase at an expression level higher relative to the wild type cell. This is advantageous as a single cell may be used to produce a higher alcohol from non-petroleum based sources such as ethanol and/or acetic acid. Also, using the recombinant cell makes the method of producing higher alcohols more efficient.

According to one aspect of the present invention, there is provided a microbial cell which is capable of producing at least one higher alcohol, wherein the cell is genetically modified to comprise an increased expression relative to its wild type cell of at least one acyl-CoA reductase (E₁₁) (EC.1.2.1.10).

The phrase “wild type” as used herein in conjunction with a cell or microorganism may denote a cell with a genome make-up that is in a form as seen naturally in the wild. The term may be applicable for both the whole cell and for individual genes. The term ‘wild type’ may thus also include cells which have been genetically modified in other aspects (i.e. with regard to one or more genes) but not in relation to the genes of interest. The term “wild type” therefore does not include such cells or such genes where the gene sequences of the specific genes of interest have been altered at least partially by man using recombinant methods. A wild type cell according to any aspect of the present invention thus refers to a cell that has no genetic mutation with respect to the whole genome and/or a particular gene. Therefore, in one example, a wild type cell with respect to enzyme E₁ may refer to a cell that has the natural/non-altered expression of the enzyme E₁ in the cell. The wild type cell with respect to enzyme E₂, E₃, E₄, E₅, E₆, E₇, E₈, E₉, E₁₀, E₁₁, E₁₂ₐ, E_{12b}, E₁₃, etc. may be interpreted the same way and may refer to a cell that has the natural/non-altered expression of the enzyme E₂, E₃, E₄, E₅, E₆, E₇, E₈, E₉, E₁₀, E₁₁, E₁₂ₐ, E_{12b}, E₁₃, etc. respectively in the cell.

A skilled person would be able to use any method known in the art to genetically modify a cell or microorganism. According to any aspect of the present invention, the genetically modified cell may be genetically modified so that in a defined time interval, within 2 hours, in particular within 8 hours or 24 hours, it forms at least twice, especially at least 10 times, at least 100 times, at least 1000 times or at least 10000 times more acetoacetate and/or 3-hydroxybutyrate than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (higher alcohol) in the nutrient medium.

The genetically modified cell or microorganism may be genetically different from the wild type cell or microorganism. The genetic difference between the genetically modified microorganism according to any aspect of the present invention and the wild type microorganism may be in the presence of a complete gene, amino acid, nucleotide etc. in the genetically modified microorganism that may be absent in the wild type microorganism. In one example, the genetically modified microorganism according to any aspect of the present invention may comprise enzymes that enable the microorganism to produce higher alcohols. The wild type microorganism relative to the genetically modified microorganism of the present invention may have none or no detectable activity of the enzymes that enable the genetically modified microorganism to produce the at least one higher alcohol. As used herein, the term ‘genetically modified microorganism’ may be used interchangeably with the term ‘genetically modified cell’.

The genetic modification according to any aspect of the present invention may be carried out on the cell of the microorganism.

The cells according to any aspect of the present invention are genetically transformed according to any method known in the art. In particular, the cells may be produced according to the method disclosed in WO/2009/077461.

The phrase ‘the genetically modified cell has an increased activity, in comparison with its wild type, in enzymes’ as used herein refers to the activity of the respective enzyme that is increased by a factor of at least 2, in particular of at least 10, more in particular of at least 100, yet more in particular of at least 1000 and even more in particular of at least 10000.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity and optionally by combining these measures. Genetically modified cells used in the method according to the invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. An “increased activity of an enzyme” may be used interchangeably with the overexpression of an enzyme.

The cell used according to any aspect of the present invention may be from a microorganism that may be capable of carrying out the ethanol-carboxylate fermentation pathway. The cell according to any aspect of the present invention may be capable of carrying out the ethanol-carboxylate fermentation pathway and may be capable of converting ethanol and/or acetate to the corresponding higher acid. The ethanol-carboxylate fermentation pathway is described in detail at least in Seedorf, H., et al., 2008. In particular, the microorganism may be selected from the group consisting of Clostridium kluyveri, C. carboxidivorans and the like. These microorganisms include microorganisms which in their wild-type form do not have an ethanol-carboxylate fermentation pathway, but have acquired this trait as a result of genetic modification. In particular, the microorganism may be Clostridium kluyveri.

In one example, the microorganism may be a wild type organism that expresses at least one enzyme selected from E₁ to E₁₀, wherein E₁ is an alcohol dehydrogenase (adh), E₂ is an acetaldehyde dehydrogenase (ald), E₃ is an acetoacetyl-CoA thiolase (thl), E₄ is a 3-hydroxybutyryl-CoA dehydrogenase (hbd), E₅ is a 3-hydroxybutyryl-CoA dehydratase (crt), E₆ is a butyryl-CoA dehydrogenase (bcd), E₇ is an electron transfer flavoprotein subunit (etf), E₈ is a coenzyme A transferase (cat), E₉ is an acetate kinase (ack) and E₁₀ is phosphotransacetylase (pta).

In particular, the wild type microorganism according to any aspect of the present invention may express at least E₂, E₃ and E₄. Even more in particular, the wild type microorganism according to any aspect of the present invention may express at least E₄.

In another example, the microorganism according to any aspect of the present invention may be a genetically modified organism that has increased expression relative to the wild type microorganism of at least one enzyme selected E₁ to E₁₀, wherein E₁ is an alcohol dehydrogenase (adh), E₂ is an acetaldehyde dehydrogenase (ald), E₃ is an acetoacetyl-CoA thiolase (thl), E₄ is a 3-hydroxybutyryl-CoA dehydrogenase (hbd), E₅ is a 3-hydroxybutyryl-CoA dehydratase (crt), E₆ is a butyryl-CoA dehydrogenase (bcd), E₇ is an electron transfer flavoprotein subunit (etf), E₈ is a coenzyme A transferase (cat), E₉ is an acetate kinase (ack) and E₁₀ is phosphotransacetylase (pta). In particular, the genetically modified microorganism according to any aspect of the present invention may express at least enzymes E₂, E₃ and E₄. Even more in particular, the genetically modified microorganism according to any aspect of the present invention may express at least E₄. The enzymes E₁ to E₁₀ may be isolated from Clostridium kluyveri.

According to any aspect of the present invention, E₁ may be an ethanol dehydrogenase. In particular, E₁ may be selected from the group consisting of alcohol dehydrogenase 1, alcohol dehydrogenase 2, alcohol dehydrogenase 3, alcohol dehydrogenase B and combinations thereof. More in particular, E₁ may comprise sequence identity of at least 50% to a polypeptide selected from the group consisting of CKL_1075, CKL_1077, CKL_1078, CKL_1067, CKL_2967, CKL_2978, CKL_3000, CKL_3425, and CKL_2065. Even more in particular, E₁ may comprise a polypeptide with sequence identity of at least 50, 60, 65, 70, 75, 80, 85, 90, 91, 94, 95, 98 or 100% to a polypeptide selected from the group consisting of CKL_1075, CKL_1077, CKL_1078 and CKL_1067.

According to any aspect of the present invention, E₂ may be an acetaldehyde dehydrogenase. In particular, E₂ may be selected from the group consisting of acetaldehyde dehydrogenase 1, alcohol dehydrogenase 2 and combinations thereof. In particular, E₂ may comprise sequence identity of at least 50% to a polypeptide selected from the group consisting of CKL_1074, CKL_1076 and the like. More in particular, E₂ may comprise a polypeptide with sequence identity of at least 50, 60, 65, 70, 75, 80, 85, 90, 91, 94, 95, 98 or 100% to a polypeptide selected from the group consisting of CKL_1074 and CKL_1076.

According to any aspect of the present invention, E₃ may be selected from the group consisting of acetoacetyl-CoA thiolase A1, acetoacetyl-CoA thiolase A2, acetoacetyl-CoA thiolase A3 and combinations thereof. In particular, E₃ may comprise sequence identity of at least 50% to a polypeptide selected from the group consisting of CKL_3696, CKL_3697, CKL_3698 and the like. More in particular, E₃ may comprise a polypeptide with sequence identity of at least 50, 60, 65, 70, 75, 80, 85, 90, 91, 94, 95, 98 or 100% to a polypeptide selected from the group consisting of CKL_3696, CKL_3697 and CKL_3698.

According to any aspect of the present invention, E₄ may be 3-hydroxybutyryl-CoA dehydrogenase 1, 3-hydroxybutyryl-CoA dehydrogenase 2 and the like. In particular, E₄ may comprise sequence identity of at least 50% to a polypeptide CKL_0458, CKL_2795 and the like. More in particular, E₄ may comprise a polypeptide with sequence identity of at least 50, 60, 65, 70, 75, 80, 85, 90, 91, 94, 95, 98 or 100% to the polypeptide CKL_0458 or CKL_2795.

According to any aspect of the present invention, E₅ may be 3-hydroxybutyryl-CoA dehydratase 1, 3-hydroxybutyryl-CoA dehydratase 2 and combinations thereof. In particular, E₅ may comprise sequence identity of at least 50% to a polypeptide selected from the group consisting of CKL_0454, CKL_2527 and the like. More in particular, E₅ may comprise a polypeptide with sequence identity of at least 50, 60, 65, 70, 75, 80, 85, 90, 91, 94, 95, 98 or 100% to a polypeptide selected from the group consisting of CKL_0454 and CKL_2527.

According to any aspect of the present invention, E₆ may be selected from the group consisting of butyryl-CoA dehydrogenase 1, butyryl-CoA dehydrogenase 2 and the like. In particular, E₆ may comprise sequence identity of at least 50% to a polypeptide selected from the group consisting of CKL_0455, CKL_0633 and the like. More in particular, E₆ may comprise a polypeptide with sequence identity of at least 50, 60, 65, 70, 75, 80, 85, 90, 91, 94, 95, 98 or 100% to a polypeptide selected from the group consisting of CKL_0455 and CKL_0633.

According to any aspect of the present invention, E₇ may be selected from the group consisting of electron transfer flavoprotein alpha subunit 1, electron transfer flavoprotein alpha subunit 2, electron transfer flavoprotein beta subunit 1 and electron transfer flavoprotein beta subunit 2. In particular, E₇ may comprise sequence identity of at least 50% to a polypeptide selected from the group consisting of CKL_3516, CKL_3517, CKL_0456, CKL_0457 and the like. More in particular, E₇ may comprise a polypeptide with sequence identity of at least 50, 60, 65, 70, 75, 80, 85, 90, 91, 94, 95, 98 or 100% to a polypeptide selected from the group consisting of CKL_3516, CKL_3517, CKL_0456 and CKL_0457.

According to any aspect of the present invention, E₈ may be coenzyme transferase (cat). In particular, E₈ may be selected from the group consisting of butyryl-CoA: acetate CoA transferase, succinyl-CoA:coenzyme A transferase, 4-hydroxybutyryl-CoA: coenzyme A transferase and the like. More in particular, E₈ may comprise sequence identity of at least 50% to a polypeptide selected from the group consisting of CKL_3595, CKL_3016, CKL_3018 and the like. More in particular, E₈ may comprise a polypeptide with sequence identity of at least 50, 60, 65, 70, 75, 80, 85, 90, 91, 94, 95, 98 or 100% to a polypeptide selected from the group consisting of CKL_3595, CKL_3016 and CKL_3018.

According to any aspect of the present invention, E₉ may be an acetate kinase A (ack A). In particular, E₉ may comprise sequence identity of at least 50% to a polypeptide sequence of CKL_1391 and the like. More in particular, E₉ may comprise a polypeptide with sequence identity of at least 50, 60, 65, 70, 75, 80, 85, 90, 91, 94, 95, 98 or 100% to a polypeptide of CKL_1391.

According to any aspect of the present invention, E₁₀ may be phosphotransacetylase (pta). In particular, E₁₀ may comprise sequence identity of at least 50% to a polypeptide sequence of CKL_1390 and the like. More in particular, E₁₀ may comprise a polypeptide with sequence identity of at least 50, 60, 65, 70, 75, 80, 85, 90, 91, 94, 95, 98 or 100% to a polypeptide of CKL_1390.

In one example the microorganism, wild-type or genetically modified expresses E₁-E₁₀. In particular, the microorganism according to any aspect of the present invention may have increased expression relative to the wild type microorganism of E₁, E₂, E₃, E₄, E₅, E₆, E₇, E₈, E₉, E₁₀ or combinations thereof. In one example, the genetically modified microorganism has increased expression relative to the wild type microorganism of E₁, E₂, E₃, E₄, E₅, E₆, E₇, E₈, E₉ and E₁₀. More in particular, a combination of any of the enzymes E₁-E₁₀ may be present in the organism to enable at least one carboxylic acid to be produced. In one example, the genetically modified organism used according to any aspect of the present invention may comprise a combination of any of the enzymes E₁-E₁₀ that enable the organism to produce at least one, or two or three types of carboxylic acids at the same time. For example, the microorganism may be able to produce hexanoic acid, butyric acid and/or acetic acid at the simultaneously. Similarly, the microorganism may be genetically modified to express a combination of enzymes E₁-E₁₀ that enable the organism to produce either a single type of carboxylic acid or a variety of carboxylic acids. In all the above cases, the microorganism may be in its wild-type form or be genetically modified.

In one example, the genetically modified microorganism according to any aspect of the present invention has increased expression relative to the wild type microorganism of hydrogenase maturation protein and/or electron transport complex protein. In particular, the hydrogenase maturation protein (hyd) may be selected from the group consisting of hydE, hydF or hydG. In particular, the hyd may comprise sequence identity of at least 50% to a polypeptide selected from the group consisting of CKL_0605, CKL_2330, CKL_3829 and the like. More in particular, the hyd used according to any aspect of the present invention may comprise a polypeptide with sequence identity of at least 50, 60, 65, 70, 75, 80, 85, 90, 91, 94, 95, 98 or 100% to a polypeptide selected from the group consisting of CKL_0605, CKL_2330 and CKL_3829.

Throughout this application, any data base code, unless specified to the contrary, refers to a sequence available from the NCBI data bases, more specifically the version online on 12 Jun. 2014, and comprises, if such sequence is a nucleotide sequence, the polypeptide sequence obtained by translating the former.

A cell according to any aspect of the present invention may be capable of producing at least one higher alcohol, wherein the cell is genetically modified to comprise an increased expression relative to its wild type cell of at least one acyl-CoA reductase (E₁₁). Acyl-CoA reductases may also be referred to as fatty acid reductases. Acyl-CoA reductases have been shown to occur in numerous kinds of organisms, including, but not limited, to bacteria, plants, fungi, algae, mammals, insects, crustaceans, and worms. Some acyl-CoA reductases, often referred to as an “alcohol-forming fatty acyl-CoA reductase” generate fatty alcohols directly via a two-step reduction as shown in Reaction [1].

Acyl-CoA+2NAD(P)H-Fatty Alcohol+2NAD(P)+ Reaction [1]

In particular, E₁₁ may be capable of catalysing the conversion of butyryl-CoA and/or hexanyl-CoA to butanol and/or hexanol respectively. The expression of E₁₁ may be measured using a method disclosed at least in Lin, 2013 or Schirmer A, 2010. In another example, “alcohol-forming fatty acyl-CoA reductase” may also include bi-functional alcohol-/aldehyde-dehydrogenases (ADH/AldDH) (EC1.1.1.1+1.2.1.10). In this example, E₁₁ may be the enzyme AdhE2 or AdhE from the microorganism selected from the group consisting of C. acetobutylicum DSM 792 (Q9ANR5), C. acetobutylicum DSM 792 (P33744), E. coli K-12 (POA9Q7), Entamoeba histolytica (Q24803), Leuconostoc mesenteroides ATCC 8293 (Q03ZS6), and C. carboxidivorans P7 (C6PZV5).

In another example, the cell according to any aspect of the present invention may be capable of producing a fatty alcohol using a two-step process as shown in Reaction [2]. Reaction [2] may be a combination of reaction (2a), (2b) or 2(c) with reaction 2(d). In one example, the cell according to any aspect of the present invention may comprise a combination of enzymes that allow for the increased reactions (2a), (2b) and/or 2(c) to lead to an increase in production of butanal relative to the wild type cell which will be used as a starting material for butanol production as shown in Reaction (2d).

Reaction [2]

acyl-CoA reductase(ACR)(E₁₁) 2(a)

Butyryl-CoA+NAD(P)H-->Butanal+CoA+NAD(P)⁺

or

carboxylic acid reductase(E₁₂ₐ) 2(b)

Butyrate+ATP+NAD(P)H-->Butanal+ADP+Pᵢ+NAD(P)⁺

or

ferredoxin oxidoreductase(AOR)(E_{12b}) 2(c)

Butyrate+Ferredoxin_{red}-->Butanal+Ferredoxinₒₓ

and

mono-functional butanol-dehydrogenase(BDH)(E₁₃) 2(d)

Butanal+NAD(P)H-->Butanol+NAD(P)⁺

The cell according to any aspect of the present invention may thus comprise an increase in expression relative to the wild type cell of enzyme E₁₁, E₁₂ₐ and/or E_{12b} and E₁₃. Examples of E₁₁, E₁₂ₐ, E_{12b} and E₁₃ are provided in table 1.

TABLE 1
Examples of enzymes used in reactions 2(a), (b), (c) and/or (d)

| | | | UniProt- | EC |
|---|---|---|---|---|
| Reaction | Enzyme | Organism | Identifier: | Nummer |
| 2(a) | Bld | Clostridium | Q7X4B7 | 1.2.1.57 |
| | | Saccheroperbutylacetonicum N1-4 | | |
| | DmpF | Pseudomonas sp. CF600 | Q52060 | 1.2.1.10 |
| | BphG | Pseudomonas cepacia | Q2VLC6 | 1.2.1.10 |
| | Ald | Clostridium beijerinckii MP | Q716S8 | 1.2.1.10 |
| 2(b) | Car | Nocadia iowensis | Q6RKB1 | 1.2.1.30 |
| | GriCD | Streptomyces griseus JCM 4626 | B1VTI3 + | 1.2.99.6 |
| | | | B1VTI2 | |
| | Car | Mycobacterium marinum ATCC BAA-535/M | B2HN69 | 1.2.1.30 |
| | Car | Streptomyces sp. W007 | H0BD51 | 1.2.1.30 |
| 2(c) | Aor | Pyrococcus furiosus DSM 3638 | Q51739 | 1.2.7.5 |
| | Aor | Clostridium acetobutylicum DSM 792 | Q97HJ3 | 1.2.7.5 |
| | Aor1 | Clostridium ljungdahlii DSM 13528 | D8GIZ8 | 1.2.7.5 |
| | Aor1 | Clostridium ljungdahlii DSM 13528 | D8GJ08 | 1.2.7.5 |
| | Aor | Clostridium carboxidivorans P7 | C6PXF4 | 1.2.7.5 |
| | Aor1 | Clostridium autoethanogenum DSM | U5RP39 | 1.2.7.5 |
| | | 10061 | | |
| | Aor2 | Clostridium autoethanogenum DSM | U5RP02 | 1.2.7.5 |
| | | 10061 | | |
| | Aor | Clostridium ragsdalei | patent | 1.2.7.5 |
| | | | only | |
| | Aor | Eubacterium limosum KIST612 | E3GFL5 | 1.2.7.5 |
| | Aor1 | Moorella thermoacetica ATCC 39073 | Q2RKJ9 | 1.2.7.5 |
| | Aor2 | Moorella thermoacetica ATCC 39073 | Q2RG52 | 1.2.7.5 |
| | Aor3 | Moorella thermoacetica ATCC 39073 | Q2RM47 | 1.2.7.5 |
| 2(d) | BdhA | Clostridium acetobutylicum DSM 792 | Q04944 | 1.1.1.1 |
| | BdhB | Clostridium acetobutylicum DSM 792 | Q04945 | 1.1.1.1 |
| | YugJ | Bacillus subtilis 168 | O05239 | 1.1.1.1 |
| | AdhC1 | Mycobacterium smegmatis ATCC | P0CH36 | 1.1.1.2 |
| | | 700084 | | |
| | AdhVII | Saccharomyces cerevisiae ATCC | P25377 | 1.1.1.2 |
| | | 204508 | | |
| | YqhD | Escherichia coli K12 | Q46856 | 1.1.1.2 |

E₁₁ may be selected from the group consisting of the acyl-CoA reductase JjFAR from the plant Simmondsia chinensis (jojoba) (Metz et al., 2000), animal acyl-CoA reductases, including those from mice, humans, and nematodes (Cheng and Russel, 2004, Moto et al., 2003). More in particular, E₁₁ may comprise an amino acid sequence that has 50, 55, 60, 65, 70, 75, 80, 85, 90, 95 or 100% sequence identity to SEQ ID NO: 1. In particular, E₁₁ may comprise an amino acid sequence SEQ ID NO: 1. E₁₁ may comprise a nucleotide sequence SEQ ID NO: 2. More in particular, E₁₁ may comprise an amino acid sequence that has 50, 55, 60, 65, 70, 75, 80, 85, 90, 95 or 100% sequence identity to SEQ ID NO: 1 and/or a nucleotide sequence that has 50, 55, 60, 65, 70, 75, 80, 85, 90, 95 or 100% sequence identity to SEQ ID NO: 2.

The higher alcohol may be selected from the group consisting of hexanol, octanol, nonanol, decanol and the like. In one example the higher alcohol may be selected from the group consisting of 1-hexanol, 2-hexanol, 3-hexanol, 1-heptanol, 2-heptanol, 3-heptanol, 4-heptanol, octanol, nananol, decanol and the like.

According to another aspect of the present invention there is provided a method of producing a higher alcohol, the method comprising

(b) contacting a recombinant microbial cell according to any aspect of the present invention with a medium comprising a carbon source A.

The term “contacting”, as used herein, means bringing about direct contact between the cell according to any aspect of the present invention and the medium comprising the carbon source. For example, the cell, and the medium comprising the carbon source may be in different compartments. In particular, the carbon source may be in a gaseous state and added to the medium comprising the cells according to any aspect of the present invention.

The carbon source A may be ethanol and/or acetate. In one example, the method according to any aspect of the present invention may comprise a first step of (a) contacting an acetogenic cell with a medium comprising a carbon source B to produce the ethanol and/or acetate of the carbon source A and the carbon source B comprises CO and/or CO₂.

The term “acetogenic bacteria” as used herein refers to a microorganism which is able to perform the Wood-Ljungdahl pathway and thus is able to convert CO, CO₂ and/or hydrogen to acetate.

These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway.

In particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990), Acetobacterium wieringae (DSM 1911), Acetobacterium woodii (DSM 1030), Alkalibaculum bacchi (DSM 22112), Archaeoglobusfulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), Clostridium ljungdahlii ERI-2 (ATCC 55380), Clostridium ljungdahlii O-52 (ATCC 55989), Clostridium mayombei (DSM 6539), Clostridium methoxybenzovorans (DSM 12182), Clostridium ragsdalei (DSM 15248), Clostridium scatologenes (DSM 757), Clostridium species ATCC 29797 (Schmidt et al., 1986), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004, Biotechnol. Let., Vol. 29, p. 1607-1612), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui). More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium ljungdahlii. In particular, strains selected from the group consisting of Clostridium ljungdahlii PETC, Clostridium ljungdahlii ERI2, Clostridium ljungdahlii COL and Clostridium ljungdahlii O-52 may be used in the conversion of synthesis gas to hexanoic acid. These strains for example are described in WO 98/00558, WO 00/68407, ATCC 49587, ATCC 55988 and ATCC 55989. In one example, both an acetogenic bacteria and the cell according to any aspect of the present invention may be used to produce a higher alcohol from the carbon source.

In one example, the acetogenic cell may be present in a first fermenter (Fermenter 1) and the cell according to any aspect of the present invention in a second fermenter (Fermenter 2). In fermenter 1, the acetogenic cells come in contact with the carbon source B to produce acetate and/or ethanol. Ethanol and/or acetate is carbon source A that may then be brought into contact with the cell according to any aspect of the present to produce at least one higher alcohol. The alcohol may then be collected and then separated from fermenter 2. A cycle may be created wherein the acetate and/or ethanol produced in fermenter 1 may be regularly fed into fermenter 2, and the acetate and/or ethanol in fermenter 2 may be converted to a higher alcohol in fermenter 2.

In another example, the media is being recycled between fermenters 1 and 2. Therefore, the ethanol and/or acetate produced in fermenter 1 may be fed into fermenter 2 and converted to the higher alcohol.

In a further example, the acetogenic cell and the cell according to any aspect of the present invention may be present in the same fermenter.

According to another aspect of the present invention, there is provided a use of the cell according to any aspect of the present invention for the production of a higher alcohol.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Generation of a Genetically Modified Clostridium kluyveri for the Formation of Higher Alcohol

The gene acyl-CoA reductase (ACR) from C. beijerinckii ATTC 35702 was codon optimized for Clostridium kluyveri and inserted into the pNW33N (AY237122.1). The vector was modified to produce plasmid pB6. Namely, PCR amplified the Gram+ origin, Gram − origin, and antibiotic markers of vector pNW33N. Both, the Gram+ and Gram − origin of replication was exchanged in the plasmid. The Gram-origin of replication was pUC 19. The CAT gene (from S. aureus plasmid pC194; Horinouchi S., 1982.) was used as the antibiotic marker for Clostridium kluyveri. The transformation of C. kluyveri was modeled after Leang et al. 2013. These sequences were transformed to be controlled by a ptb promotor. The created vector was named pB6-ACR_Cb(CoC1). The vector pB6-ACR_Cb(CoC1) was then used to modify C. kluyveri using a method compared to Leang et al. 2013. The modified C. kluyveri strain is named C. kluyveri pB6-ACR_Cb(CoC1).

### Example 2

Genetically Modified Clostridium kluyveri Forming Butanol Acid from Acetate and Ethanol

For the biotransformation of ethanol and acetate to butanol the bacterium Clostridium kluyveri pB6-ACR_Cb(CoC1) was used. All cultivation steps were carried out under anaerobic conditions in pressure-resistant glass bottles that can be closed airtight with a butyl rubber stopper. For the butanol production culture 100 ml of DMSZ 52 medium (pH=7.0; 10 g/L K-acetate, 0.31 g/L K₂HPO₄, 0.23 g/L KH₂PO₄, 0.25 g/l NH₄Cl, 0.20 g/l MgSO₄×7H₂O, 1 g/L yeast extract, 0.50 mg/L resazurin, 10 μl/l HCl (25%, 7.7 M), 1.5 mg/L FeCl₂×4H₂O, 70 g/L ZnCl₂×7H₂O, 100 g/L MnCl₂×4H₂O, 6 g/L H₃BO₃, 190 g/L CoCl₂×6H₂O, 2 g/L CuCl₂×6H₂O, 24 g/L NiCl₂×6H₂O, 36 g/L Na₂MO₄×2H₂O, 0.5 mg/L NaOH, 3 g/L Na₂SeO₃×5H₂O, 4 g/L Na₂WO₄×2H₂O, 100 g/L vitamin B12, 80 g/L p-aminobenzoic acid, g/L D(+) Biotin, 200 g/L nicotinic acid, 100 g/L D-Ca-pantothenate, 300 g/L pyridoxine hydrochloride, 200 μg/l thiamine-HCl×2H₂O, 20 ml/L ethanol, 2.5 g/L NaHCO₃, 0.25 g/L cysteine-HCl×H₂O, 0.25 g/L Na₂S×9H₂O) and 7.5 mg/L Thiamphenicol in a 250 ml bottle were inoculated with 5 ml of a frozen cryoculture of Clostridium kluyveri pB6-ACR_Cb(CoC1). This growing culture was incubated anaerobically at 37° C. for 237 h. At the start and end of the culturing period, samples were taken. These were tested for optical density, pH and the different analytes (analyzed via NMR).

The results showed that the amount of acetate decreased from 10 g/l to 2 g/l and the amount of ethanol decreased from 15.8 g/l to 8.6 g/l. Also, the concentration of butyric acid was increased from 0 g/l to 2.4 g/l and the concentration of hexanoic acid was increased from 0 g/l to 6.3 g/l. In difference to the C. kluyveri pB6 bearing the empty vector (control strain) the C. kluyveri pB6-ACR_Cb(CoC1) produces also 0.06 g/L butanol and 0.08 g/L hexanol.

### Example 3

Generation of a Genetically Modified Clostridium kluyveri for the Formation of Higher Alcohol

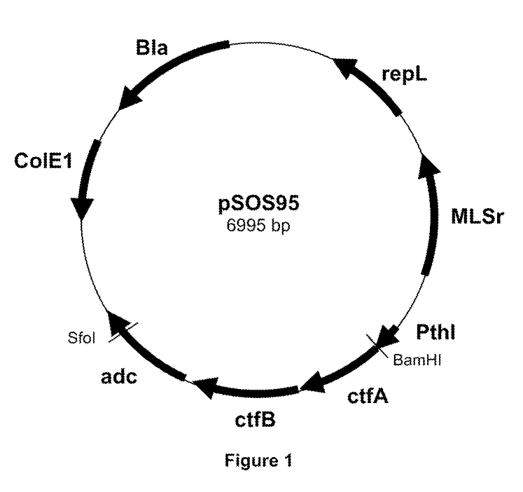

The gene acyl-CoA reductase (ACR) from C. beijerinckii ATTC 35702 will be codon optimized for Clostridium kluyveri and be inserted into the vector pEmpty. This plasmid is based on the plasmid backbone pSOS95 (FIG. 1). VectorpSOS95, will be digested with BamHI and KasI. This will remove the operon ctfA-ctfB-adc, but will leave the thl promoter and the rho-independent terminator of adc. The created vector will be named pNW95-ACR_Cb(CoC1). The vector pNW95-ACR_Cb(CoC1) will then be used to modify C. kluyveri using a method taught in Leang et al. 2013. The modified C. kluyveri strain will be named C. kluyveri pNW95-ACR_Cb(CoC1).

### Example 4

Genetically Modified Clostridium kluyveri Forming Butanol Acid from Acetate and Ethanol

For the biotransformation of ethanol and acetate to butanol the bacterium Clostridium kluyveri pNW95-ACR_Cb(CoC1) will be used. All cultivation steps will be carried out under anaerobic conditions in pressure-resistant glass bottles that can be closed airtight with a butyl rubber stopper.

For the butanol production culture 100 ml of DMSZ 52 medium (pH=7.0; 10 g/L K-acetate, 0.31 g/L K₂HPO₄, 0.23 g/L KH₂PO₄, 0.25 g/l NH₄Cl, 0.20 g/l MgSO₄×7H₂O, 1 g/L yeast extract, 0.50 mg/L resazurin, 10 μl/l HCl (25%, 7.7 M), 1.5 mg/L FeCl₂×4H₂O, 70 g/L ZnCl₂×7H₂O, 100 g/L MnCl₂×4H₂O, 6 g/L H₃BO₃, 190 g/L CoCl₂×6H₂O, 2 g/L CuCl₂×6H₂O, 24 g/L NiCl₂×6H₂O, 36 g/L Na₂MO₄×2H₂O, 0.5 mg/L NaOH, 3 g/L Na₂SeO₃×5H₂O, 4 g/L Na₂WO₄×2H₂O, 100 g/L vitamin B12, 80 g/L p-aminobenzoic acid, g/L D(+) Biotin, 200 g/L nicotinic acid, 100 g/L D-Ca-pantothenate, 300 g/L pyridoxine hydrochloride, 200 μg/l thiamine-HCl×2H₂O, 20 ml/L ethanol, 2.5 g/L NaHCO₃, 0.25 g/L cysteine-HCl×H₂O, 0.25 g/L Na₂S×9H₂O) and 7.5 mg/L Thiamphenicol in a 250 ml bottle will be inoculated with 5 ml of a frozen cryoculture of Clostridium kluyveri pNW95-ACR_Cb(CoC1). This growing culture will be anaerobically incubated at 37° C. for 237 h. At the start and end of the culturing period, samples will be taken. These will be tested for optical density, pH and the different analytes (analyzed via NMR).

The results will show that the amount of acetate and ethanol would decrease. Also, the concentration of butyric acid and hexanoic acid will increase. Compared to the C. kluyveri pEmpty bearing the empty vector (control strain) the C. kluyveri pNW95-ACR_Cb(CoC1) will produce butanol and hexanol.

### REFERENCES

Cheng and Russel, J Biol Chem 279: 37789-97 (2004)
Drake et al., 2004. Strict and Facultative Anaerobes: Medical and Environmental Aspects. pp. 251-281, Horizon Scientific Press, United Kingdom
Drake & Kusel, 2005 Acetogenic clostridia. In: Dfirre, P. (ed.), Handbook on Clostridia, pp. 719-746. CRC Press, Boca Raton, Fla.
Drake et al., 2006, Acetogenic prokaryotes. In: Balows A, Trüper H G, Dworkin M, Harder W and
Horinouchi S., J Bacteriol. 1982 May; 150(2): 815-825
Leang et al., Appl. Environ. Microbiol., 79 (4) (2013), pp. 1102-1109
Lin F., FEBS J. 2013 October; 280(19):4773-81
Metz et al., Plant Physiol 122: 635-644 (2000)
Morinaga et al., 1990, J. Biotechnol., Vol. 14, p. 187-194
Moto et al., Proc Natl Acad Sci USA 100: 9156-61 (2003)
Perez et al., Biol. Chem. 2008, 283.12:7346-7353
Schirmer A, (2010) Science 329, 559-562
Schmidt et al., 1986, Chem. Eng. Commun., Vol. 45, p. 61-73
Seedorf et al., Proc. Natl. Acad. Sci. USA 2008, 105:2128-2133
Wood, 1991 Life with CO or CO2 and H2 as a source of carbon and energy. FASEB J. 5:156-163
U.S. 2007/0275447, U.S. 2008/0057554, WO2009100434, WO/2009/077461

1. A microbial cell capable of producing at least one higher alcohol, wherein the microbial cell is genetically modified to comprise an increased expression relative to its wild type cell of at least one acyl-CoA reductase (E₁₁) and wherein the microbial cell is capable of producing a carboxylic acid and/or ester thereof using ethanol-carboxylate fermentation.

2. The microbial cell according to claim 1, wherein the microbial cell expresses at least one enzyme selected from the group consisting of alcohol dehydrogenase E₁, acetaldehyde dehydrogenase E₂, acetoacetyl-CoA thiolase E₃, 3-hydroxybutyryl-CoA dehydrogenase E₄, 3-hydroxybutyryl-CoA dehydratase E₅, butyryl-CoA dehydrogenase E₆, electron transfer flavoprotein subunit E₇, coenzyme A transferase E₈, acetate kinase E₉, phosphotransacetylase E₁₀.

3. The microbial cell according claim 1, wherein the microbial cell is from a microorganism selected from the group consisting of Clostridium kluyveri and C. carboxidivorans.

4. The microbial cell according to claim 1, wherein acyl-CoA reductase (E₁₁) is capable of catalysing Reaction 1 and/or Reaction 2(a) below:
Acyl-CoA+2NAD(P)H→Fatty Alcohol Reaction 1:
Butyryl-CoA+NAD(P)H-->Butanal+CoA+NAD(P)⁺. Reaction 2(a):

5. The microbial cell according to claim 1, wherein E₁₁ is from Clostridium beijerinckii.

6. The microbial cell according to claim 1, wherein E₁₁ comprises 60% sequence identity with SEQ ID NO: 1.

7. The microbial cell according to claim 1, wherein the microbial cell is further genetically modified to comprise an increased expression relative to its wild type cell of mono-functional butanol-dehydrogenase (BDH) (E₁₃).

8. The microbial cell according to claim 7, wherein the microbial cell is further genetically modified to comprise an increased expression relative to its wild type cell of carboxylic acid reductase (E₁₂ₐ), ferredoxin oxidoreductase (AOR) (E_{12b}) and/or mono-functional butanol-dehydrogenase (BDH) (E₁₃)

9. The microbial cell according to claim 1, wherein the microbial cell expresses hydrogenase maturation protein and/or electron transport complex protein.

10. The microbial cell according to claim 1, wherein the microbial cell is genetically modified to comprise increased expression relative to the wild type cell of at least one enzyme selected from the group consisting of E₂, E₃, E₄, E₅, E₆, E₇, E₈, E₉, E₁₀, hydrogenase maturation protein and/or electron transport complex protein.

11. The microbial cell according to claim 1, wherein the higher alcohol is butanol and/or hexanol.

12. A method of producing a higher alcohol, the method comprising the step of:
(b) contacting a recombinant microbial cell according to claim 1 with a medium comprising a carbon source A wherein the carbon source A comprises ethanol and/or acetate.

13. The method according to claim 12, wherein the method further comprises
(a) contacting an acetogenic cell with a medium comprising a carbon source B to produce the ethanol and/or acetate of the carbon source A and the carbon source B comprises CO and/or CO₂.

14. The method according to claim 12, wherein the higher alcohol is selected from the group consisting of 1-hexanol, 2-hexanol, 3-hexanol, 1-heptanol, 2-heptanol, 3-heptanol, 4-heptanol, octanol, nananol, decanol.

15. (canceled)

16. The method according to claim 13, wherein the higher alcohol is selected from the group consisting of 1-hexanol, 2-hexanol, 3-hexanol, 1-heptanol, 2-heptanol, 3-heptanol, 4-heptanol, octanol, nananol, decanol.

17. The microbial cell according claim 2, wherein the microbial cell is from a microorganism selected from the group consisting of Clostridium kluyveri and C. carboxidivorans.

18. The microbial cell according to claim 2, wherein acyl-CoA reductase (E₁₁) is capable of catalysing Reaction 1 and/or Reaction 2(a) below:
Acyl-CoA+2NAD(P)H-Fatty Alcohol Reaction 1:
Butyryl-CoA+NAD(P)H-->Butanal+CoA+NAD(P)⁺. Reaction 2(a):

19. The microbial cell according to claim 2, wherein E₁₁ is from Clostridium beijerinckii.

20. The microbial cell according to claim 2, wherein E₁₁ comprises 60% sequence identity with SEQ ID NO: 1.

21. The microbial cell according to claim 2, wherein the microbial cell is further genetically modified to comprise an increased expression relative to its wild type cell of mono-functional butanol-dehydrogenase (BDH) (E₁₃).


SEQUENCE LISTING (2 sequences)

SEQ ID NO: 1 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 2 - DNA, 1407 bp, Clostridium beijerinckii
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa